(19) United States
(12) Patent Application Publication
(10) Pub. No.: US 2025/0128046 A1
(43) Pub. Date: Apr. 24, 2025

(54) MECHANICAL CIRCULATORY SUPPORT SYSTEM WITH REPOSITIONING SHEATH

(71) Applicants: Boston Scientific Scimed, Inc., Maple Grove, MN (US); Boston Scientific Medical Device Limited, Galway (IE)

(72) Inventors: Qian Liu, Plymouth, MN (US); Neeraj Kumar Sharma, Delhi (IN)

(73) Assignees: Boston Scientific Scimed, Inc., Maple Grove, MN (US); Boston Scientific Medical Device Limited, Galway (IE)

(21) Appl. No.: 18/917,595

(22) Filed: Oct. 16, 2024

Related U.S. Application Data
(60) Provisional application No. 63/591,819, filed on Oct. 20, 2023.

(51) Int. Cl. A61M 60/289

(52) U.S. Cl. CPC A61M 60/289

(57) ABSTRACT

A mechanical circulatory support system may include a blood pump, a housing, an elongate shaft, a first sheath, and a second sheath. The blood pump may pump blood from a ventricle of a heart of a patient to vasculature of the patient. The elongate shaft may couple with the blood pump and extend proximally from the blood pump to the housing. The first sheath may have a first hub and a lumen with an inner diameter. The second sheath may have a second hub and an outer diameter that is less than the inner diameter of the lumen. The first sheath and the second sheath may longitudinally advance over the elongate shaft and a distal end of the second sheath may advance through the lumen and extend distally beyond a distal end of the first sheath.

### CROSS-REFERENCE TO RELATED APPLICATIONS

The present application claims the benefit of U.S. Provisional Patent Application Ser. No. 63/591,819, filed Oct. 20, 2023, the disclosure of which is incorporated herein by reference.

### TECHNICAL FIELD

The present disclosure pertains to a sheath configuration for a mechanical circulatory support system. More particularly, the present disclosure pertains to a repositioning sheath of a mechanical circulatory support system for use at a vascular access site.

### BACKGROUND

In various procedures for delivering intravascular medical devices, an introducer sheath is inserted into a blood vessel of a patient, for example a femoral artery, and one or more medical devices may be advanced through the sheath and into the patient's vasculature. In various instances, the medical devices include catheters or other devices, such as a blood pump. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including percutaneous blood pumps and associated devices.

A first example may include a mechanical circulatory support system comprising a blood pump configured to pump blood from a ventricle of a heart of a patient to vasculature of the patient, a housing, an elongate shaft coupled with the blood pump and extending proximally from the blood pump to the housing, a first sheath having a first hub, the first sheath has a lumen with an inner diameter, and a second sheath having a second hub, the second sheath has an outer diameter that is less than the inner diameter of the lumen, and wherein the first sheath and the second sheath may be configured to longitudinally advance over the elongate shaft and a distal end of the second sheath may be configured to advance through the lumen of the first sheath and extend distally beyond a distal end of the first sheath.

Alternatively or additionally to any of the examples above, the first sheath may be an introducer sheath configured to allow the blood pump to pass through the lumen.

Alternatively or additionally to any of the examples above, the second sheath may be a dual lumen sheath.

Alternatively or additionally to any of the examples above, the dual lumen sheath may comprise a first tube defining a first lumen, a second tube extending along the first tube and defining a second lumen, and a third tube extending over the first tube and the second tube.

Alternatively or additionally to any of the examples above, the system may further include a wire, and the dual lumen sheath may define a first lumen configured to receive the elongate shaft and a second lumen configured to receive the wire.

Alternatively or additionally to any of the examples above, the second sheath may include an internal hemostasis valve.

Alternatively or additionally to any of the examples above, the first hub may be configured to couple with the second hub when the distal end of the second sheath is distal of the distal end of the first sheath.

Alternatively or additionally to any of the examples above, the first hub may include a suture pad configured to detach from the first hub and slide over the first sheath to a location around the second sheath when the distal end of the second sheath is distal of the distal end of the first sheath.

Alternatively or additionally to any of the examples above, the system may further include a clip configured to couple the suture pad with the second sheath when the suture pad is at the location around the second sheath.

Alternatively or additionally to any of the examples above, the second hub may include an adjustable shaft lock configured to releasably secure the second hub relative to the elongate shaft.

In a further example, a method of adjusting a tube assembly for a mechanical circulatory support system may comprise advancing a blood pump and an elongate shaft extending proximally from the blood pump through a first sheath, advancing a distal end of a second sheath over the elongate shaft and through a distal end of the first sheath to a location distal of a distal end of the first sheath, and coupling a first hub of the first sheath with a second hub of the second sheath.

Alternatively or additionally to any of the examples above, the method may further include detaching a suture pad from the first hub and positioning the suture pad at a location around the second sheath at a location distal of the distal end of the first sheath.

Alternatively or additionally to any of the examples above, the method may further include coupling the suture pad with the second sheath using a clip.

Alternatively or additionally to any of the examples above, the method may further include locking the second sheath to the elongate shaft.

Alternatively or additionally to any of the examples above, the method may further include adjusting a position of the blood pump by engaging the elongate shaft through a sleeve coupled with the second hub and advancing the elongate shaft relative to the second sheath.

Alternatively or additionally to any of the examples above, advancing the distal end of the second sheath may include advancing the distal end of the second sheath to a location distal of a patient access site.

Alternatively or additionally to any of the examples above, the method may further include withdrawing a distal end of the first sheath from the access site when the distal end of the second sheath is at the location distal of the patient access site.

Alternatively or additionally to any of the examples above, withdrawing a distal end of the first sheath from the access site may include advancing the first hub over the second sheath toward the second hub for coupling with the second hub.

In a further example, a mechanical circulatory support system may comprise a blood pump configured to pump blood from a ventricle of a heart of a patient to vasculature of the patient, an elongate shaft coupled with the blood pump and extending proximally from the blood pump, an introducer sheath having a first hub and configured to extend over the elongate shaft, the introducer sheath has a lumen with an inner diameter configured to receive the blood pump, and a repositioning sheath having a second hub and configured to extend over the elongate shaft, the repositioning sheath has an outer diameter that is less than the inner diameter and a length that is longer than a length of the introducer sheath, wherein the first hub may be configured to couple with the second hub when a distal end of the repositioning sheath is distal of a distal end of the introducer sheath.

Alternatively or additionally to any of the examples above, the first hub may include a detachable suture pad configured to slide over the introducer sheath to a location around the repositioning sheath when the distal end of the repositioning sheath is distal of the distal end of the introducer sheath.

The above summary of some embodiments is not intended to describe each disclosed embodiment or every implementation of the present disclosure. The Figures and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative catheter system including a percutaneous blood pump;

FIG. 2 is a schematic partial cross-section view of a proximal end of an illustrative catheter system, in a first configuration;

FIG. 3 is a schematic partial cross-section view of the proximal end of the illustrative catheter system depicted in FIG. 1, in a second configuration;

FIG. 4 is a schematic cross-section view of an illustrative repositioning sheath;

FIG. 5 is a schematic cross-section view of an illustrative repositioning sheath with a hub and positioned on an elongate shaft;

FIGS. 6A-6F depict a schematic flow of an illustrative technique for adjusting a tube assembly of a catheter system; and

FIGS. 7A-7B depict a schematic flow of an illustrative technique for adjusting a tube assembly of a catheter system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

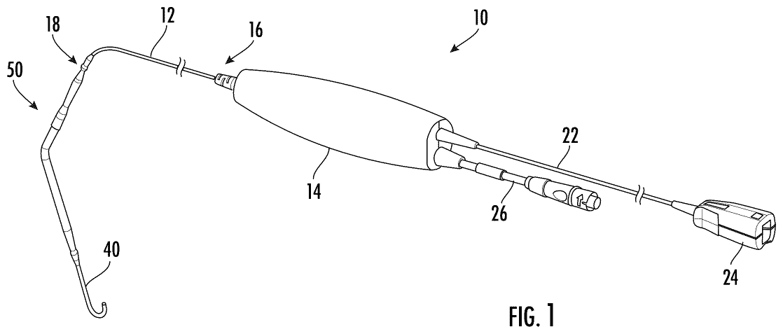

FIG. 1 illustrates a perspective view of a catheter 10 (e.g., a mechanical circulatory support system and/or other suitable catheter system) including a percutaneous blood pump 50 located at a distal end region thereof. The blood pump 50 may be configured to pump blood from a ventricle of a heart of a subject (e.g., a patient) to vasculature of the subject and/or between other portions of the circulatory system.

The catheter 10 may be coupled to or include the blood pump 50, with an elongate shaft 12 (e.g., an elongate tube with one or more elongate members extending therein) of the catheter 10 extending proximally from the percutaneous blood pump 50 and a distal tip 40 extending distally from the blood pump 50. For instance, a proximal end 16 of the elongate shaft 12 may be coupled to a housing 14 (e.g., a housing of a handle, of a control module, etc.) and a distal end 18 of the elongate shaft 12 may be coupled to the percutaneous blood pump 50. An electrical cable 22 may extend from the housing 14 to a connector 24 at a proximal end thereof. The connector 24 may be configured to be connected to a controller (not shown) for controlling the blood pump 50, such as providing electrical power, control signals, etc. to the blood pump 50. The catheter 10 may also include an extension 26 connectable to the controller for sending and/or receiving signals, such as from one or more sensors during operation of the blood pump 50.

When placing the blood pump 50 in a subject (e.g., a patient), the blood pump 50 may be inserted through an introducer sheath inserted into a vascular access site of the subject. In some cases, the introducer sheath may be a large bore sheath configured to receive and facilitate the passage therethrough of the blood pump 50 and the elongate shaft 12. In some cases, such an introducer sheath may have an outer diameter that tends to block the free flow of blood through the femoral artery to a leg of the subject.

To facilitate perfusion of blood to the leg of the subject, the introducer sheath may be swapped out for a smaller outer diameter sheath, such as a repositioning sheath. To facilitate removal of the introducer sheath from the vascular access site, the introducer sheath may be a peel-away sheath, where the introducer sheath may be configured to split along a perforated line extending entirely along or at least part of a length of the introducer sheath, and/or may be removable from the catheter 10 in one or more other suitable manners.

Peel-away introducer sheaths may be undesirable for a variety of reasons. In some examples, peel-away introducer sheaths may unintentionally or uncontrollably peel, which may lead to vessel trauma. In some examples, peel-away introducer sheaths may be formed from a polymer, which may require a thicker wall than other materials (e.g., metals) used for forming the introducer sheath to create a wall that has a similar strength to sheaths made out of the other materials. As such, when a polymer peel-away introducer sheath is used, a larger hole or access site in the subject may be required for receiving the introducer sheath relative to when a non-peel-away introducer sheath is utilized.

The concepts discussed herein include utilizing an introducer sheath and a repositioning sheath that are both configured to receive and advance over the elongate shaft 12. In some configurations, the repositioning sheath may have a length that is longer than a length of the introducer sheath such that a distal end of the repositioning sheath may be inserted into an access site to a location distal of a distal end of the introducer sheath before, during, or after the introducer sheath is fully withdrawn from the access site, while a proximal hub of the repositioning sheath is proximal of the proximal hub of the introducer sheath.

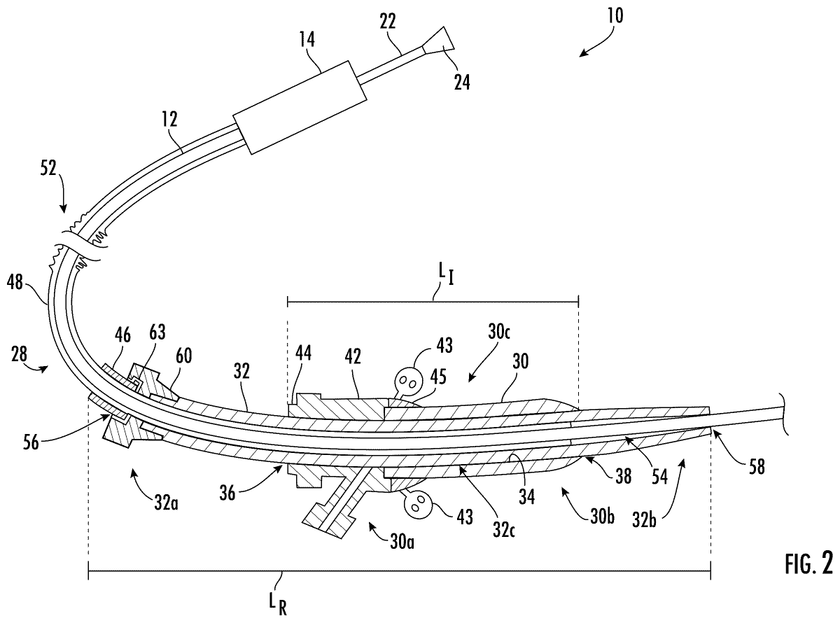

FIG. 2 depicts a schematic partial cross-section view of an illustrative configuration of a proximal end of the catheter 10. The proximal end of the catheter 10 may include the elongate shaft 12 extending distally from the housing 14 through a sterile sleeve 28, an introducer sheath 30 (e.g., a first sheath), and a repositioning sheath 32 (e.g., a second sheath), wherein the sterile sleeve 28, the introducer sheath 30, and the repositioning sheath 32 are depicted in cross-section.

In some cases, the introducer sheath 30 may be used for facilitating passage of various medical devices, such as the elongate shaft 12, the blood pump 50, etc., through an access site of the subject and into a blood vessel of the subject. The introducer sheath 30 may include a proximal end region 30a proximate a proximal end of the introducer sheath 30 and a distal end region 30b proximate a distal end of the introducer sheath 30. A body region 30c of the introducer sheath 30 may extend between the proximal end region 30a and the distal end region 30b, where one or more of the proximal end region 30a, the distal end region 30b, and the body region 30c may define a lumen 34 of the introducer sheath 30. Further, the introducer sheath 30 may include a proximal opening 36 at or proximate the proximal end region 30a and a distal opening 38 at or proximate the distal end region 30b, with the lumen 34 extending between the proximal opening 36 and the distal opening 38. The lumen 34 may define an inner diameter of the introducer sheath 30.

The introducer sheath 30 may be formed from one or more suitable materials. Example suitable materials include, but are not limited to, polymeric and/or metallic materials. In some instances, the introducer sheath 30 may include one or more additional surface coatings on the interior surface defining the lumen 34 and/or the exterior surface, which may include, but are not limited to, silicone, polyethylene terephthalate (PET), and/or other suitable coating materials.

The introducer sheath 30 (e.g., at the proximal end region thereof) may include a hub 42 (e.g., a proximal hub, such as a hemostasis valve hub, and/or other suitable hub) that may provide access to the lumen 34. The hub 42 may be configured for hemostasis by, for example, preventing or mitigating blood leakage out of the introducer sheath 30 during use. For example, a medical device, such as the elongate shaft 12 and/or the blood pump 50, may be inserted through the hub 42 and the lumen 34 of the introducer sheath 30 and into the blood vessel of the subject, and the hub 42 may maintain hemostasis between the medical device, the introducer sheath 30, and the external surroundings.

After positioning the medical device at a target site within the subject, fixation of the axial and radial position of the medical device may be desired to ensure that the medical device (and any device coupled thereto) is maintained in a proper position while in use. It may also be desired for the medical personnel to reposition the medical device after insertion. As such, the hub 42 may include a tightening port 44, provided with or attachable thereto, that provides for the fixation of the medical device (e.g., the elongate shaft 12) with respect to the hub 42. Further, to affix the introducer sheath 30 relative to the access site of the subject, the hub 42 may include or may be coupled to one or more suture pads 43 (e.g., two suture pads 43 extending from the hub 42, as depicted in FIG. 2) that may be sutured and/or otherwise coupled to a skin surface of the subject.

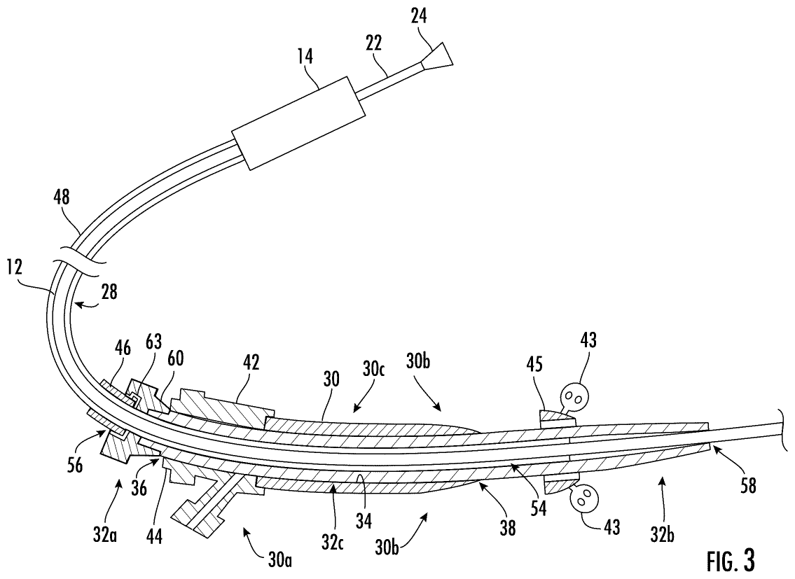

The suture pads 43 may be separable from other portions of the hub 42. In some examples, the suture pads 43 may include or may be coupled with a suture pad ring 45 that is releasably coupled with the hub 42 (e.g., as depicted in FIG. 3) and configured to advance along the introducer sheath 30 and couple with the repositioning sheath 32 when disconnected from the hub 42.

The suture pad ring 45 may be coupled with the hub 42 in any suitable manner. Example suitable coupling mechanisms include, but are not limited to, a luer connection, a clip, a clasp, a friction fit connection, a rotational tightening mechanism, threads, and/or other suitable types of coupling mechanisms. In some examples, the suture pad ring 45 may be configured to couple with the hub 42 using a friction fit connection, but this is not required. The coupling mechanisms may be integral with the suture pad ring 45 and/or separate from the suture pad ring 45.

The suture pad ring 45 may be coupled with the repositioning sheath 32 in any suitable manner. Example suitable coupling mechanisms include, but are not limited to, a luer connection, a clip, a clasp, a friction fit connection, a rotational tightening mechanism, threads, and/or other suitable type of coupling mechanism. In some examples, the suture pad ring 45 may be configured to couple with repositioning sheath 32 using a clip connection, but this is not required. The coupling mechanisms may be integral with the suture pad ring 45 and/or separate from the suture pad ring 45.

The tightening port 44 of the hub 42 may be any suitable type of tightening port. For example, the tightening port 44 may engage the elongate shaft 12 in response to rotating a rotational actuator of the tightening port 44, adjusting a clip of the tightening port 44, and/or the tightening port 44 may engage the elongate shaft 12 with one or more additional or alternative coupling techniques discussed herein or otherwise. Further, in some cases, the tightening port 44 and/or other suitable portions of the hub 42 may be configured to receive and couple with a hub 60 (e.g., a proximal hub) of the repositioning sheath 32 and/or other portions of the repositioning sheath 32.

The introducer sheath 30 may have any suitable length L_{I}. In some examples, the length L_{I} of the introducer sheath 30 may extend from a proximal end of the introducer sheath 30 at or proximate the proximal opening 36 to a distal end of the introducer sheath 30 at or proximate the distal opening 38. Example suitable lengths of the introducer sheath 30 include, but are not limited to, lengths in a range of about thirteen centimeters (cm) to about thirty three cm.

The repositioning sheath 32 may be configured to replace the introducer sheath 30 at the access site and may be configured to advance over the elongate shaft 12 and through the introducer sheath 30 to the access site of the subject. For example, once the blood pump 50 has been positioned in the blood vessel of the subject and/or at a target site within the subject, the repositioning sheath 32 (e.g., a distal end of the repositioning sheath 32) may be advanced toward the access site through the introducer sheath 30 to a location distal of a distal end of the introducer sheath 30 and positioned across the access site and in the blood vessel of the subject. The repositioning sheath 32 may be inserted into the access site and the blood vessel of the subject before, during, and/or after the introducer sheath 30 has been removed from the access site.

When positioned in the blood vessel of the subject, the repositioning sheath 32 may facilitate repositioning of the blood pump 50 at or proximate the target site within the subject through advancing of the elongate shaft 12 extending into the access site of the subject and the blood vessel via the repositioning sheath 32. The repositioning sheath 32 may facilitate increased blood perfusion through the blood vessel relative to the blood perfusion that occurs when the introducer sheath 30 is in the blood vessel due to the repositioning sheath 32 having a smaller outer diameter than the introducer sheath 30. Further, the repositioning sheath 32 may be less stiff or rigid than the introducer sheath 30, which may reduce trauma to the subject at the access site and mitigate effects that movements of the catheter 10 proximal of the access site have on a position of the blood pump 50 at the target site.

The repositioning sheath 32 may include a proximal end region 32a proximate a proximal end of the repositioning sheath 32 and a distal end region 32b proximate a distal end region of the repositioning sheath 32. A body region 32c of the repositioning sheath 32 may extend between the proximal end region 32a and the distal end region 32b, where one or more of the proximal end region 32a, the distal end region 32b, and the body region 32c may define a lumen 54 of the repositioning sheath 32. Further, the repositioning sheath 32 may include a proximal opening 56 at or proximate the proximal end region 32a and a distal opening 58 at or proximate the distal end region 32b, with the lumen 54 extending between the proximal opening 56 and the distal opening 58.

The repositioning sheath 32 may have any suitable configuration between the proximal end region 32a and the distal end region 32. In some examples, the outer diameter of the repositioning sheath 32 may change along a length of the repositioning sheath 32. In one example, the distal end region 32b of the repositioning sheath 32 may have a smaller outer diameter than the proximal end region 32a and/or the body region 32c, where the outer diameter of the repositioning sheath 32 may taper distally between or at the body portion 32c and the distal end region 32b. The taper or reduced outer diameter at the distal end region 32b may facilitate mitigating leg ischemia due to a relatively small outer diameter portion of the repositioning sheath 32 being located in the vessel of the subject and/or may facilitate reinserting the repositioning sheath 32 through an access site and into the subject.

The repositioning sheath 32 may have any suitable number of lumens. Although FIG. 2 depicts only the single lumen 54 of the repositioning sheath 32, the repositioning sheath 32 may have a single lumen, two lumens, three lumens, and/or other suitable number of lumens as desired.

The repositioning sheath 32 may have any suitable length L_{R}. In some examples, the length L_{R} of the repositioning sheath 32 may extend from a proximal end of the repositioning sheath 32 at or proximate the proximal opening 56 to a distal end of the repositioning sheath 32 at or proximate the distal opening 58. Example suitable lengths of the repositioning sheath 32 include, but are not limited to, lengths in a range of about thirteen centimeters (cm) to about forty cm. The length L_{R} of the repositioning sheath 32 may be configured to be longer than the length L_{I} of the introducer sheath 30, such that a distal end of the repositioning sheath 32 is configured to advance through the access site into a blood vessel, while allowing the introducer sheath 30 to be advanced proximally (e.g., withdrawn) from the access site over the repositioning sheath 32 such that the hub 60 of the repositioning sheath 32 may couple with or be positioned proximate the hub 42 of the introducer sheath 30.

The repositioning sheath 32 may be formed in any suitable manner. In some examples, the repositioning sheath 32 may be formed with one or more extrusion processes and/or other suitable manufacturing processes. When the repositioning sheath 32 is formed with more than one lumen, the different lumens may be formed by combining multiple tubes within an outer tube, but this is not required and other configurations for forming multiple lumens are contemplated. In one example, when the repositioning sheath 32 includes two lumens, a first lumen may be formed from a first tube, a second lumen may be formed from a second tube, and a third tube may surround the first tube and the second tube to form the outer surface and/or diameter of the repositioning sheath 32. The tubes of the repositioning sheath 32, when multiple tubes are utilized, may be coupled using any suitable coupling technique including, but not limited to, adhesives, reflow techniques, and/or other suitable coupling techniques.

The repositioning sheath 32 may be formed from one or more suitable materials. Example suitable materials include, but are not limited to, polymeric and/or metallic materials. In some instances, the repositioning sheath 32 may include one or more additional surface coatings on the interior surface defining the lumen 54 and/or the exterior surface, which may include, but are not limited to, silicone, polyethylene terephthalate (PET), and/or other suitable coating materials.

The repositioning sheath 32 may have any suitable outer diameter that is smaller than the outer diameter of the introducer sheath 30 so as to better allow blood perfusion through the blood vessel of the subject in which the elongate shaft 12 is inserted and reduce a maintained diameter opening at the access site of the subject while the blood pump 50 is positioned at a target site (e.g., during a dwell time). In some examples, the outer diameter of the repositioning sheath 32 may be less than an inner diameter of the introducer sheath 30 to facilitate passing the repositioning sheath 32 through the introducer sheath 30, but this is not required and the introducer sheath 30 may be configured to stretch or flex radially outward to allow the repositioning sheath 32 to pass through the introducer sheath 30.

A hub 60 may be at and/or coupled to a proximal end of the repositioning sheath 32 and may be considered part of the repositioning sheath 32, but this is not required. The hub 60 may facilitate the proximal opening 56 of the repositioning sheath 32 and may facilitate receiving and/or coupling with one or more components of the catheter 10.

In some cases, the hub 60 may include one or more coupling mechanisms configured to couple with the elongate shaft 12 (e.g., an elongate shaft lock) to facilitate releasably fixing the elongate shaft 12 relative to the repositioning sheath 32. Example suitable coupling mechanisms include, but are not limited to, a clip, a clasp, a friction fit connection, a rotational tightening mechanism, threads, and/or other suitable type of coupling mechanism. In some examples, the hub 60 may include a rotational tightening coupling mechanism configured to couple with the elongate shaft 12 at a desired location along the elongate shaft 12.

The hub 60 of the repositioning sheath 32 may include a port 64 for receiving and/or coupling with a head 46 of the sterile sleeve 28. In some examples, the port 64 may be configured to receive the head 46 of the sterile sleeve 28 within the lumen 54 to couple the sterile sleeve 28 with the repositioning sheath 32 (e.g., via a friction fit connection, a luer connection, a clip or clasp connection, threads, and/or other suitable type of connection) and create a sterile field along the elongate shaft 12 between the proximal end of the repositioning sheath 32 and the distal end of the housing 14 during a medical procedure.

The sterile sleeve 28 may extend from the housing 14 distally over the elongate shaft 12. In some configurations of the sterile sleeve 28, the sterile sleeve 28 may include the distal head 46 and a tubular member 48 extending proximally from the head 46 to the housing 14. The tubular member 48 of the sterile sleeve 28 may be flexible and configured to have bunched locations 52 when there is slack in the tubular member 48 between the head 46 of the sterile sleeve 28 and the housing 14, as depicted, for example, in FIG. 2.

The tubular member 48 of the sterile sleeve 28 may be formed from any suitable materials. Example suitable material for forming the tubular member 48 include, but are not limited to, polyurethane, thermoplastic polyurethane (TPU), polyethylene, ethylene vinyl acetate (EVA), and/or other suitable materials.

FIG. 3 depicts the catheter 10 with the repositioning sheath 32 advanced distally through the introducer sheath 30 and/or the introducer sheath 30 is advanced proximally over the repositioning sheath 32, such that a portion (e.g., at least a distal end) of the repositioning sheath 32 distal of the distal end of the introducer sheath 30 is a sufficient length to pass through the access site of the subject and into the blood vessel of the subject. In some examples and as depicted in FIG. 3, the hub 60 of the repositioning sheath 32 may abut the hub 42 of the introducer sheath 30 when the repositioning sheath 32 is advanced distally relative to the introducer sheath 30. Although not required, the hub 60 of the repositioning sheath 32 may be configured to be secured to the hub 42 of the introducer sheath 30.

As depicted in FIG. 3, the suture pad ring 45 may be separated from a portion of the hub 42 fixed with the introducer sheath 30 and advanced distally over the introducer sheath 30 and the repositioning sheath 32. The suture pad ring 45 may then be coupled with the repositioning sheath 32 at a location distal of the distal end of the introducer sheath 30. The suture pad ring 45 may couple with the hub 42 and the repositioning sheath 32 using the same coupling technique or mechanism or different coupling techniques or mechanisms, as desired. In one example, the suture pad ring 45 may couple with at least the repositioning sheath 32 using a clip connection.

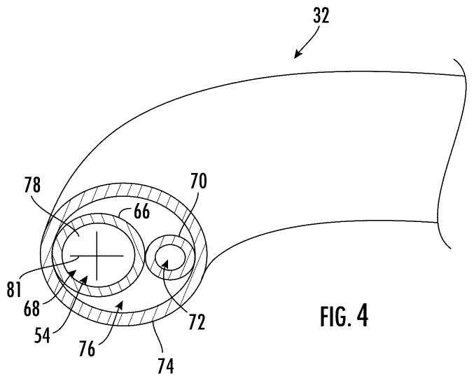

FIG. 4 depicts a schematic cross-sectioned view of an illustrative configuration of the repositioning sheath 32 independent of the other components of the catheter 10. Although not required, the repositioning sheath 32 may be a dual lumen sheath with a first tube 66 defining a first lumen 68 (e.g., the first lumen 68 may be the lumen 54, discussed above, that is configured to receive the elongate shaft 12 of the catheter 10), a second tube 70 extending along the first tube 66 and defining a second lumen 72 configured to receive one or more wires, and a third tube 74 extending over the first tube 66 and the second tube 70. Other suitable configurations of the repositioning sheath 32 are contemplated. In some cases, the third tube 74 may define a third lumen 76, but this is not required, and the third tube 74 may be configured to fit over and fill gaps (e.g., via heat shrinking, etc.) around the first tube 66 and the second tube 70.

The first tube 66, the second tube 70, and/or the third tube 74 may be formed from one or more suitable materials. Example suitable materials include, but are not limited to, polymeric and/or metallic materials. In some instances, the first tube 66, the second tube 70, and/or the third tube 74 may include one or more additional surface coatings on the interior surfaces defining the lumens 68, 72, 76 and/or the exterior surface, which may include, but are not limited to, silicone, polyethylene terephthalate (PET), and/or other suitable coating materials.

The first lumen 68 may include a hemostasis valve 78 (e.g., an internal hemostasis valve) extending across the first lumen 68 (e.g., the lumen 54 configured to receive the elongate shaft 12) to prevent blood from flowing through the first lumen 68 and out of the repositioning sheath 32. Additionally or alternatively, the second lumen 72 may include a hemostasis valve (not shown).

The hemostasis valve 78 may be any suitable type of valve configured to block blood flow through, for example, the first lumen 68. In some examples, the hemostasis valve 78 may be or and/or may be formed from a silicone disc with a cross-cut 81 configured to receive the elongate shaft 12 and allow the repositioning sheath 32 to pass over and seal around the elongate shaft 12. However, it is contemplated that the hemostasis valve 78 may be formed from one or more additional or alternative materials and/or include one or more other suitable cuts or openings configured to receive and seal around the elongate shaft 12.

FIG. 5 depicts a schematic cross-sectioned view of the illustrative configuration of the repositioning sheath 32 depicted in FIG. 4, with components of the catheter 10. For example, the repositioning sheath 32 may include the hub 60 and the elongate shaft 12 may extend through the first lumen 68 and the hemostasis valve 78.

The sterile sleeve 28 may extend over the elongate shaft 12 to create a sterile boundary over the elongate shaft 12 from the hub 60 to the housing 14 (not shown in FIG. 5). In some examples, the head 46 of the sterile sleeve 28 may couple with the hub 60, as discussed above.

The hub 60 may be configured to receive a stylet 80 having a wire 82 and a cap 84 at a port, such as a side port 71 and/or at one or more other suitable ports. The stylet 80 may be inserted into the hub 60 and the repositioning sheath 32 such that the wire 82 may extend through the second lumen 72 (e.g., where the side port 71 may be in communication with the second lumen 72), but this is not required and the wire 82 may extend through one or more other suitable lumens. When the wire 82 of the stylet 80 extends through the second lumen 72, the wire 82 may be configured to prevent the second lumen 72 from becoming blocked by coagulated blood, but this is not required.

The cap 84 of the stylet 80 may be configured to couple with the hub 60 (e.g., with a proximal end of the side port 71) using any suitable adjustable or separable coupling techniques and/or mechanisms. Example suitable coupling techniques and/or mechanisms include, but are not limited to, a luer connection, a clip, a clasp, a friction fit connection, a rotational tightening mechanism, threads, and/or other suitable types of coupling mechanisms.

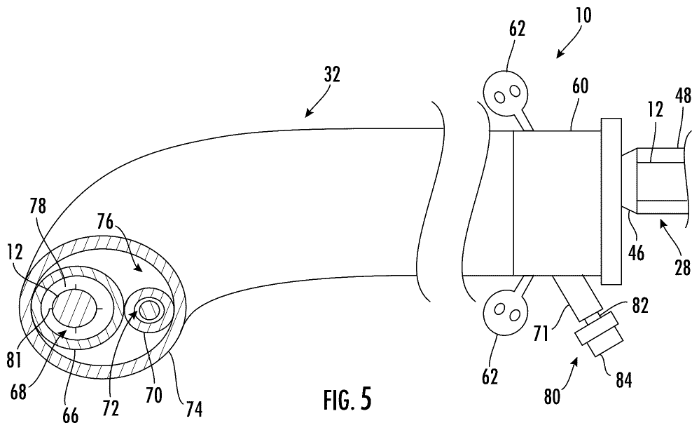

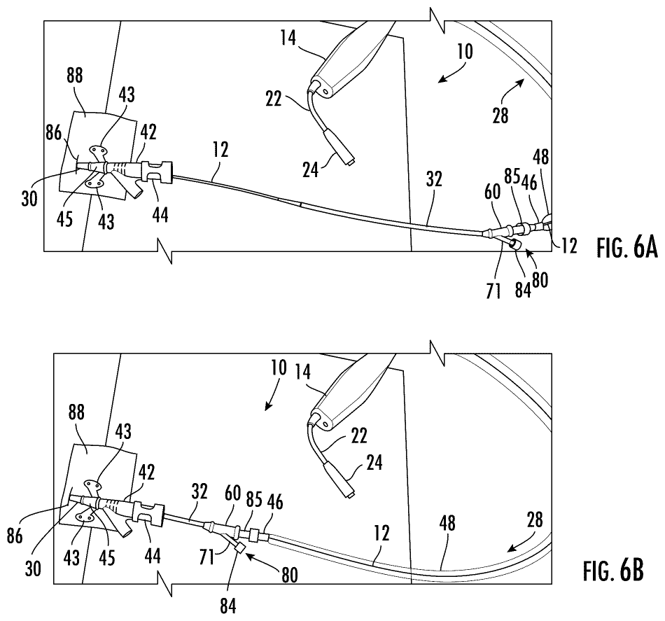

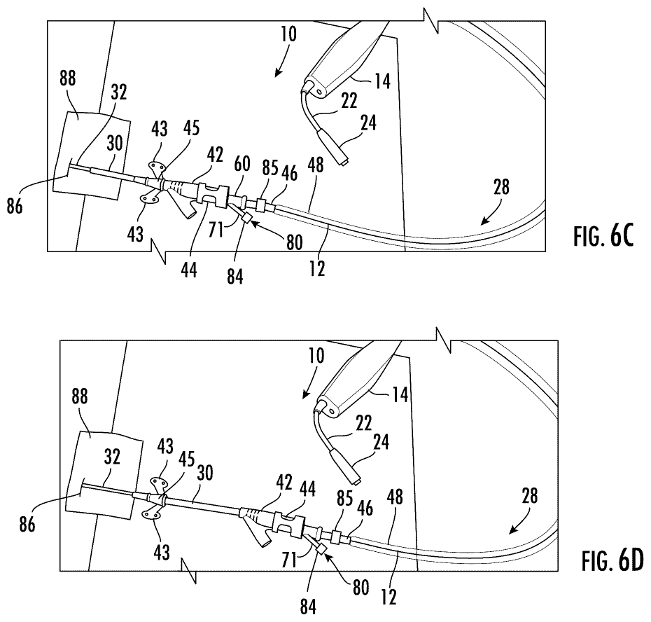

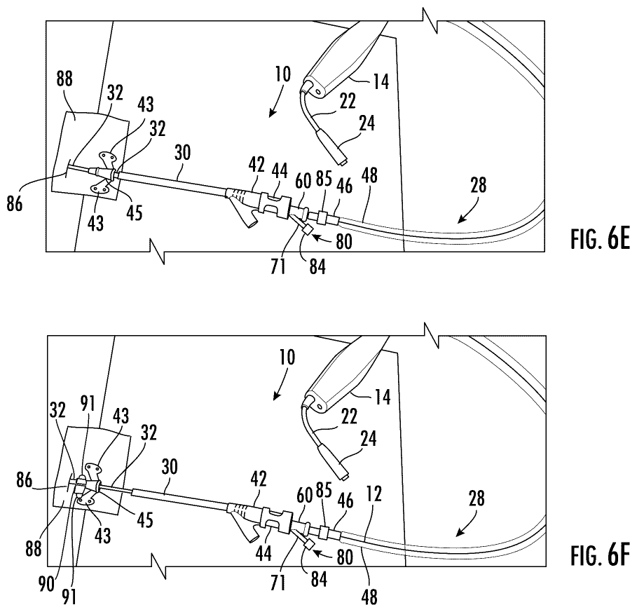

FIGS. 6A-6F schematically depict an illustrative technique for replacing the introducer sheath 30 with the repositioning sheath 32 at an access site 86 in a subject 88. Although the illustrative technique for replacing the introducer sheath 30 with the repositioning sheath 32 is discussed with respect to the components of the catheter 10, other suitable components may be utilized as desired.

FIG. 6A schematically depicts the catheter 10 inserted into the access site 86 of the subject 88, with the elongate shaft 12 extending proximal from the proximal hub 42 of the introducer sheath 30, through the repositioning sheath 32 and the sterile sleeve 28. During advancing (e.g., insertion) of the blood pump 50 and the elongate shaft 12 through the introducer sheath 30 and into the subject 88, the repositioning sheath 32 and the sterile sleeve 28 may remain proximal of the introducer sheath 30. In some cases, the sterile sleeve 28 may be coupled to the hub 60 and the hub 60 may be coupled with or fixed to the elongate shaft 12 via an elongate shaft lock 85 in a locked position or configuration, but this is not required.

FIG. 6B schematically depicts the introducer sheath 30 positioned in the access site 86, with the repositioning sheath 32 advanced distally along the elongate shaft 12 and into the introducer sheath 30. Prior to advancing the repositioning sheath 32 along the elongate shaft 12, the elongate shaft lock 85 of or proximate the hub 60 may be adjusted from a locked position or configuration to an unlocked position or configuration to disengage the elongate shaft lock 85 from the elongate shaft 12. Once the elongate shaft lock 85 is in an unlocked position or configuration, a user may advance the distal end of the repositioning sheath 32 into the introducer sheath 30 to a location across the access site 86 and distal of the distal end of the introducer sheath 30, and into the blood vessel, but the repositioning sheath 32 may be advanced distally in one or more other manners. Further, the sterile sleeve 28 may be advanced distally with the repositioning sheath 32, as depicted in FIG. 6B, but this is not required and the repositioning sheath 32 may be advanced distally into the introducer sheath 30 and the subject 88 independent of the sterile sleeve 28. Advancing the repositioning sheath 32 into the introducer sheath 30 and/or the subject 88 may occur with the introducer sheath 30 in the access site 86, but this is not required and the introducer sheath 30 may be removed from the access site 86 prior to, during, or after removing the introducer sheath 30 from the access site 86.

FIG. 6C schematically depicts the introducer sheath 30 advanced proximally from the access site 86 (e.g., fully withdrawn from the access site 86 with the distal end of the introducer sheath 30 proximal of the access site 86) and the repositioning sheath 32 extending into access site 86 (e.g., with the distal end of the repositioning sheath 32 is distal of the distal end of the introducer sheath 30). When advancing the introducer sheath 30 proximally, the hub 42 may advance over the repositioning sheath 32 to a location at or proximate to the hub 60 of the repositioning sheath 32 for coupling therewith.

If the suture pads 43 of the hub 42 are sutured to the subject 88, the sutures may be removed from the suture pads 43 and/or the subject 88 prior to withdrawing the introducer sheath 30 from the access site 86. Alternatively, the suture pads 43 may be disengaged from the hub 42 and remain sutured in place while the introducer sheath 30 is withdrawn from the access site 86.

When the introducer sheath 30 has been fully withdrawn from the access site 86, the hub 42 of the introducer sheath 30 may couple with or engage the hub 60 of the repositioning sheath 32. In some examples, the hub 42 may releasably couple with the hub 60 in any suitable manner discussed herein or otherwise to fix the introducer sheath 30 relative to the repositioning sheath 32.

FIG. 6D schematically depicts the suture pad ring 45 and the suture pads 43 detached or separated from the hub 42 of the introducer sheath 30 and advanced distally toward the access site 86 (e.g., when the suture pads 43 were advanced proximally with the introducer sheath 30). The suture pads 43 may be separated from the other portions of the hub 42 and/or the introducer sheath 30 and advanced over the introducer sheath 30 toward the access site 86 to facilitate suturing the suture pads 43 to the subject 88 at a location over the repositioning sheath 32 and/or proximate the access site 86. The suture pad ring 45 may be detached from the introducer sheath 30 and/or the hub 42 in any suitable manner discussed herein or otherwise. However, when the suture pads 43 do not detach from the other components of the hub 42, the suture pads 43 may be sutured to the subject at or proximate the hub 42.

FIG. 6E schematically depicts the suture pads 43 and the suture pad ring 45 proximate the access site and positioned distal of the distal end of the introducer sheath 30 and along the repositioning sheath 32. When positioned over the repositioning sheath 32 distal of the introducer sheath 30, the suture pad ring 45 may be adjusted to couple with the repositioning sheath 32 proximal of and proximate the access site 86.

The suture pad ring 45 may be coupled with the repositioning sheath 32 in any suitable manner discussed herein or otherwise. In some examples, the suture pad ring 45 may couple with the repositioning sheath 32 in a similar manner to how the suture pad ring 45 may be coupled with the introducer sheath 30 and/or the hub 42. In one example, a clip or clasp of or separate from the suture pad ring 45 may be used to couple the suture pad ring 45 with the repositioning sheath 32, but other configurations are contemplated.

Once proximate the access site 86, the suture pads 43 may be sutured to the subject 88. Suturing of the suture pads 43 to the subject may occur before, during, or after coupling the suture pad ring 45 with the repositioning sheath 32, as desired.

FIG. 6F schematically depicts a clip 90 (e.g., a suture pad clip or other suitable clip) coupled with the suture pad ring 45 and the repositioning sheath 32. In some instances, when the suture pad ring 45 does not include a coupling mechanism and/or in other instances, the clip 90 may be utilized to couple the repositioning sheath 32 with or relative to the suture pad ring 45 when the suture pad ring 45 is located at or around the repositioning sheath 32.

In some examples, the clip 90 may include wings 91 (e.g., two wings and/or other suitable number of wings). In operation, the wings 91 may be compressed toward each other when loading the clip 90 onto the repositioning sheath 32 and the suture pad ring 45. The wings 91 may then be released to cause the clip 90 to engage the repositioning sheath 32 with the repositioning sheath 32 and the suture pad ring 45 and fix the repositioning sheath 32 and the suture pad ring 45 relative to one another. Other suitable clips 90 are contemplated.

Further, the elongate shaft lock 85 of the hub 60 of the repositioning sheath 32 may be adjusted from an unlocked position or configuration to a locked position or configuration to secure the hub 60, along with one or more of the sterile sleeve 28, the introducer sheath 30, and the repositioning sheath 32 to or relative to the elongate shaft 12. The fixing of the hub 60 relative to the elongate shaft 12 may occur at any suitable time after positioning the repositioning sheath 32 through the access site 86 and into the blood vessel of the subject 88.

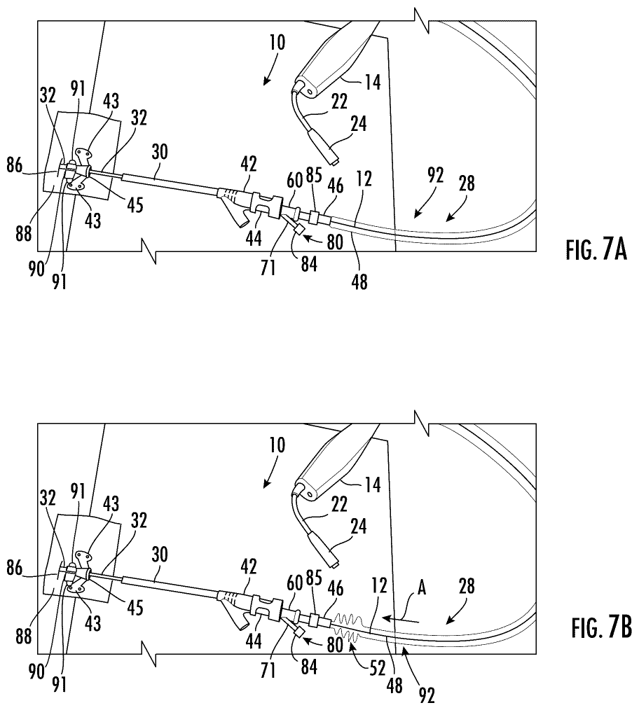

FIGS. 7A and 7B schematically depict an illustrative technique for repositioning the blood pump 50 (not shown) by advancing the elongate shaft 12 proximal of the repositioning sheath 32. Although the illustrative technique for repositioning the blood pump 50 is discussed with respect to the components of the catheter 10, other suitable components may be utilized as desired.

FIG. 7A schematically depicts the sterile sleeve 28 coupled with the hub 60 at the proximal end of the repositioning sheath 32. In preparation for adjusting a position of the blood pump 50, the elongate shaft lock 85 of or at the hub 60 may be opened or adjusted to an unlocked position or configuration such that the elongate shaft 12 may adjust positions relative to sterile sleeve 28, the introducer sheath 30, and/or the repositioning sheath 32. Further, a grasp location 92 along the sterile sleeve 28 and the elongate shaft 12 at which the elongate shaft 12 may be grasped through the sterile sleeve 28 for adjusting the blood pump 50 is depicted in FIG. 7A.

FIG. 7B schematically depicts the elongate shaft 12 and the sterile sleeve 28 advanced (e.g., adjusted) distally in the direction of arrow A. In the example adjustment of the elongate shaft 12 depicted in FIG. 7B to adjust a position of the blood pump 50, a user may grasp the elongate shaft 12 through the tubular member 48 (e.g., the flexible tubular member) of the sterile sleeve 28 at the grasp location 92 and advance both of the elongate shaft 12 and the tubular member 48 distally, which results in advancing the blood pump 50 within the subject 88 and creating a bunched location 52 in the tubular member 48 distal of the grasp location 92. The blood pump 50 may be adjusted in a proximal direction by advancing the elongate shaft 12 and the tubular member 48 in the proximal direction. Once the blood pump 50 and/or the elongate shaft 12 has been adjusted to a desired location, the elongate shaft lock 85 at or of the hub 60 may be adjusted to a locked position or configuration to secure the elongate shaft 12 relative to the sterile sleeve 28, the introducer sheath 30, and/or the repositioning sheath 32.

Although not depicted, catheter 10 may be utilized in one or more other techniques and/or methods including, but not limited to, techniques for removing the blood pump 50 from the subject 88 and/or techniques for reinserting the introducer sheath 30 after the introducer sheath 30 has been removed from the access site 86. Although illustrative technique for removing the blood pump 50 and/or for reinserting the introducer sheath 30 into the access site are described below with respect to the components of the catheter 10, other suitable components may be utilized as desired and/or other suitable blood pump techniques may be utilized.

In the illustrative removal technique, a user may withdraw the stylet 80 from the hub 60 of the repositioning sheath 32. In some examples, the cap 84 may be disconnected (e.g., unscrewed, etc.) from the hub 60 and advanced (e.g., withdrawn) in a direction away from the hub 60 to remove the wire 82 of the stylet 80 from the repositioning sheath 32 (e.g., from the second lumen 72 of the repositioning sheath 32).

A syringe may be positioned at a lumen (e.g., the second lumen 72) of the repositioning sheath 32 in which the wire 82 was previously positioned so as to be in fluid communication with the lumen. In some cases, the syringe may couple with the hub 60 in the same or similar manner as the stylet 80. With the syringe, a user may flush (e.g., with suction) the lumen to remove any debris therein. Although the syringe is described as being used to flush the lumen, other suitable components may be utilized to flush the lumen including, but not limited to, an electromechanical suction device and/or other suitable device.

A wire may be inserted into the hub 60 of the repositioning sheath 32. In some examples, the wire may be inserted into the hub 60, into the lumen (e.g., the second lumen 72) of the repositioning sheath 32 in which the wire 82 of the stylet 80 was located, through the repositioning sheath 32 and the access site 86, and into the subject 88 (e.g., in a blood vessel in which the elongate shaft 12 is located). In some cases, the wire may remain in the subject 88 at the access site 86 after the blood pump 50 has been removed to facilitate one more additional treatments or procedures if any are needed after the blood pump 50 has been removed from the subject 88. The wire may be utilized for other suitable purposes, as desired.

The wire may have any suitable diameter. In some examples, the wire may have diameter of 0.035 inches (or 0.889 millimeters), but wires with other suitable diameters are contemplated. The wire may be a guide wire, but other suitable wires are contemplated.

Once the wire has been placed within the subject, an elongate shaft lock 85 of or at the hub 60 may be adjusted to an unlock position or configuration and the elongate shaft 12 may be advanced in a proximal direction relative to the repositioning sheath 32 to move the blood pump 50 into the descending aorta of the subject 88, while maintaining a position of the wire in the subject 88. Moving the blood pump 50 into the descending aorta of the subject 88 may occur before, during, or after removing the repositioning sheath 32 from the access site 86. In some examples, the elongate shaft lock 85 may remain in a locked configuration and the repositioning sheath 32 may be withdrawn from the access site 86 as the elongate shaft 12 is advanced proximally to move the blood pump 50 into the descending aorta, while maintaining a position of the wire 96 in the subject 88, but this is not required.

When the repositioning sheath 32 has been fully removed from the access site 86, the wire may be held in place within the access site 86 (e.g., via tape, a user, etc.) and a user may fully withdraw the elongate shaft 12 and the blood pump 50 from the subject 88. After removing the blood pump 50 from the user, the access site 86 may be closed around the wire to prevent leakage of body fluids (e.g., blood). Closing the access site 86 may include suturing the access site closed around the wire and/or one or more other suitable closing techniques.

In some examples, it may be desirable to reinsert the introducer sheath 30 into the access site 86 after the introducer sheath 30 has been removed from the access site 86. Reinserting the introducer sheath 30 into the access site 86 may facilitate exchanging blood pumps used to treat the subject 88 and/or performing one other interventions.

In one example of reinserting the introducer sheath 30 into the access site 86 after the introducer sheath 30 has been removed from the access site 86, the introducer sheath 30 may be reinserted into the access site 86 while the repositioning sheath 32 is extending within the access site 86. When the repositioning sheath 32 is extending through the access site 86 and into the subject 88, the introducer sheath 30 may be advanced distally over the repositioning sheath 32 and into the access site 86 to a location within the vessel of the subject 88. Although not required, the introducer sheath 30 may be advanced through the suture pads 43 as the introducer sheath 30 is advanced over the repositioning sheath 30. When the clip 90 is coupled to the repositioning sheath 32 and the suture pads 43, the clip 90 may be removed prior to reinserting the introducer sheath 30 into the subject 88. After reinserting the introducer sheath 30, the repositioning sheath 32 and the blood pump 50 may be removed from the subject 88 through the lumen 34 of the introducer sheath 30. Then a new blood pump 50 and/or other suitable device may be inserted through the lumen 34 of the introducer sheath 30, across the access site 86, and into the vessel of the subject 88 for use in one or more interventions and/or procedures.

In an additional or alternative example of reinserting the introducer sheath 30 into the access site 86 (e.g., and into a vessel of the subject 88) after the introducer sheath 30 has been removed from the access site 86, the introducer sheath 30 may be reinserted into the access site 86 with the repositioning sheath 32. In some examples, as discussed above and/or otherwise, the repositioning sheath 32 may be removed from the subject over a wire inserted therethrough and the wire may remain in the access site 86 of the subject 88. When the wire is extending from the subject 88 via the access site 86 and/or in other instances, the repositioning sheath 32 and the introducer sheath 30 may be reinserted into the access site 86 by advancing distally into the access site 86, where the repositioning sheath 32 may track over the wire. Before, during, or after positioning the introducer sheath 30 in the vessel of the subject 88, the repositioning sheath 32 may be advanced proximally out of the access site 86. In some examples, the wire extending through the access site 86 may also be advanced proximally to withdraw the wire from the subject 88 before, during, or after the introducer sheath 30 is inserted in the vessel of the subject 88. After reinserting the introducer sheath 30, the blood pump 50 may be removed from the subject 88 through the lumen 34 of the introducer sheath 30. Then a new blood pump 50 and/or other suitable device may be inserted through the lumen 34 of the introducer sheath 30, across the access site 86, and into the vessel of the subject 88 for use in one or more interventions and/or procedures.

The schematic methods and techniques discussed herein may be used together, unless expressly indicated otherwise. Further, the order of implementation of the steps discussed herein may be in other suitable orders, unless expressly indicated otherwise, may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A mechanical circulatory support system, the system comprising:
a blood pump configured to pump blood from a ventricle of a heart of a patient to vasculature of the patient;
a housing;
an elongate shaft coupled with the blood pump and extending proximally from the blood pump to the housing;
a first sheath having a first hub, the first sheath has a lumen with an inner diameter; and
a second sheath having a second hub, the second sheath has an outer diameter that is less than the inner diameter of the lumen, and
wherein the first sheath and the second sheath are configured to longitudinally advance over the elongate shaft and a distal end of the second sheath is configured to advance through the lumen of the first sheath and extend distally beyond a distal end of the first sheath.

2. The system of claim 1, wherein the first sheath is an introducer sheath configured to allow the blood pump to pass through the lumen.

3. The system of claim 1, wherein the second sheath is a dual lumen sheath.

4. The system of claim 3, wherein the dual lumen sheath comprises a first tube defining a first lumen, a second tube extending along the first tube and defining a second lumen, and a third tube extending over the first tube and the second tube.

5. The system of claim 3, further comprising:
a wire, and
wherein the dual lumen sheath defines a first lumen configured to receive the elongate shaft and a second lumen configured to receive the wire.

6. The system of claim 1, wherein the second sheath includes an internal hemostasis valve.

7. The system of claim 1, wherein the first hub is configured to couple with the second hub when the distal end of the second sheath is distal of the distal end of the first sheath.

8. The system of claim 1, wherein the first hub includes a suture pad configured to detach from the first hub and slide over the first sheath to a location around the second sheath when the distal end of the second sheath is distal of the distal end of the first sheath.

9. The system of claim 8, further comprising:
a clip configured to couple the suture pad with the second sheath when the suture pad is at the location around the second sheath.

10. The system of claim 1, wherein the second hub includes an adjustable shaft lock configured to releasably secure the second hub relative to the elongate shaft.

11. A method of adjusting a tube assembly for a mechanical circulatory support system, the method comprising:
advancing a blood pump and an elongate shaft extending proximally from the blood pump through a first sheath;
advancing a distal end of a second sheath over the elongate shaft and through a distal end of the first sheath to a location distal of a distal end of the first sheath; and
coupling a first hub of the first sheath with a second hub of the second sheath.

12. The method of claim 11, further comprising:
detaching a suture pad from the first hub; and
positioning the suture pad at a location around the second sheath at a location distal of the distal end of the first sheath.

13. The method of claim 12, further comprising:
coupling the suture pad with the second sheath using a clip.

14. The method of claim 11, further comprising:
locking the second sheath to the elongate shaft.

15. The method of claim 11, further comprising:
adjusting a position of the blood pump by engaging the elongate shaft through a sleeve coupled with the second hub and advancing the elongate shaft relative to the second sheath.

16. The method of claim 11, wherein advancing the distal end of the second sheath includes advancing the distal end of the second sheath to a location distal of a patient access site.

17. The method of claim 16, further comprising:
withdrawing a distal end of the first sheath from the access site when the distal end of the second sheath is at the location distal of the patient access site.

18. The method of claim 17, wherein withdrawing a distal end of the first sheath from the access site includes advancing the first hub over the second sheath toward the second hub for coupling with the second hub.

19. A mechanical circulatory support system, the system comprising:
a blood pump configured to pump blood from a ventricle of a heart of a patient to vasculature of the patient;
an elongate shaft coupled with the blood pump and extending proximally from the blood pump;
an introducer sheath having a first hub and configured to extend over the elongate shaft, the introducer sheath has a lumen with an inner diameter configured to receive the blood pump; and
a repositioning sheath having a second hub and configured to extend over the elongate shaft, the repositioning sheath has an outer diameter that is less than the inner diameter and a length that is longer than a length of the introducer sheath, and
wherein the first hub is configured to couple with the second hub when a distal end of the repositioning sheath is distal of a distal end of the introducer sheath.

20. The system of claim 19, wherein the first hub includes a detachable suture pad configured to slide over the introducer sheath to a location around the repositioning sheath when the distal end of the repositioning sheath is distal of the distal end of the introducer sheath.
